(19) United States
(12) Patent Application Publication
(10) Pub. No.: US 2016/0106877 A1
(43) Pub. Date: Apr. 21, 2016

(54) Air Fragrance Dispersion Device

(71) Applicants: Paul Joseph Albee, Lambertville, NJ (US); Eric Matthew Albee, Lambertville, NJ (US); Sandra Albee Keeley, Bensalem, PA (US)

(72) Inventors: Paul Joseph Albee, Lambertville, NJ (US); Eric Matthew Albee, Lambertville, NJ (US); Sandra Albee Keeley, Bensalem, PA (US)

(21) Appl. No.: 14/517,819

(22) Filed: Oct. 18, 2014

(51) Int. Cl. A61L 9/12

(52) U.S. Cl. CPC A61L 9/122

(57) ABSTRACT

A fragrance diffusing apparatus, comprises a hollow housing consisting of: a top dispersion cap with at least one outlet and a motorized fan disposed therein contiguously joined to a central body portion via an internal cavity formed by a cowling having a plurality of first inlet vents and at least one outlet portal connected to said dispersion cap. The device also comprises a lower base portion connected to said central body portion and the base contains an electric power source for running said motorized fan wherein all three sections are connected as an integrated unit. The motorized fan is positioned within the cowling so as to provide a strong up-draft of air through the central cavity and a ventilated fragrance tray in juxtaposition thereto to force-ably disperse the fragrance throughout the surrounding atmosphere.

### I. FIELD OF THE INVENTION

The present invention relates generally to room air fresheners and, more particularly, fragrance air diffusers for scenting a room or other enclosed area. The invention disperses one of a number of scents or fragrances from a housing having a plurality of outlet and inlet vents, a chamber for holding a fragrance medium, and a fan for drawing air from the inlet vents and creating an updraft airflow which facilitates the emission of the fragrance from the fragrance medium.

### II. BACKGROUND OF THE INVENTION

Air diffusers, also known as air fresheners, have existed for some time. Generally, an air freshener emits an aroma into a room or enclosed area. The aroma may create a mood, invoke a psychological response and/or mask unpleasant odors. In some instances, aromas are used for therapeutic purposes. For example, aromatherapy is a form of alternative medicine that employs the aromas of volatile essential oils for the purpose of affecting a person's mood or health, mentally, emotionally, and spiritually. Essential oils are concentrated aromatic oils obtained from plants, flowers, and herbs. Different oils have different therapeutic effects when the aromas are inhaled. The most common way of using essential oils in aromatherapy is by dispersing the aroma via various diffusing systems. As stated above, such systems may also be used to mask unpleasant odors.

Previous methods of diffusing essential oils used candle diffusers. Candle diffusers typically include a candleholder in the lower part of the diffuser and a reservoir above the candle to contain the oil. A candle, typically a tea-light, is placed in the candleholder to heat the oil above, causing the oil to evaporate into the air. However, candle diffusers are dangerous to use because of the risk of fire, and therefore should not be left unattended.

Electric diffusers that are inexpensive to manufacture and which diffuse fragrances into the air without the risk of fire have become available. These electric diffusers receive power from either a household electrical outlet, or a battery. They generally comprise a receptacle for containing the fragrance oil and an electric heater that heats the oil causing it to evaporate into the air. These diffusers are capable of dispersing fragrance continuously into the air, while providing the benefit of remaining unattended, without the risk of starting a fire.

A common problem with heat-activated diffusers (i.e., candle diffusers, electric diffusers, etc.) is that the smell of the produced fragrance is altered as essential oils are burned when heated. Burned oils may incur health hazards, since some burned components in the essential oils may be toxic. The burning of the oils may also alter the beneficial effects of the oils. Thus, cold air diffusers are generally preferred.

A nebulizer is one form of a cold air diffuser. It consists of a base containing a motor, whereby the base is constructed from blown-glass. The nebulizer breaks the essential oil into separate molecules before dispersing them into the air. Although this method is said to have high therapeutic value, nebulizers are expensive and are difficult to maintain. The blown-glass piece is easily breakable and expensive to replace. In addition, some nebulizers are incompatible with oils that have heavy particle sizes.

An alternative method of dispersing fragrance oils without heat is by using fan-based electric diffusers. Typically, these systems comprise a housing, an air inlet and an air outlet, a battery-operated fan, and a fragrance source containing having either a liquid fragrance concentrate or a fragrance emitting solid. When turned on the fan forces air past the fragrance containing liquid or solid thereby creating a scented airflow between the air inlet and the air outlet. The airflow causes the evaporated liquid in the container to travel through the diffuser, whereby the fragrance is finally emitted into the surrounding air from the diffuser via the air outlet.

Electrical devices to evaporate a perfume and/or fragrance composition into a space, particularly a domestic space, such as a living room, to provide a pleasant aromas are well known in the art. There are a variety of such devices on sale, for example, the device commercially available as Airwick®, the air fragrance diffused Actif® (Reckitt Benckiser), Renusit®, etc. Generally, these devices consist of a perfume or fragrance source, an electrical heater, a capillary element or substrate from which the perfume is vaporized by application of heat, and a power supply. This arrangement provides a continuous supply of the perfume to the space in which the device is placed. However, it may take longer to fragrance a room, because this arrangement relies on vaporization involving the generation of smaller molecules that diffuse quickly and are more easily absorbed into surrounding objects.

U.S. Pat. No. 6,413,476 discloses and claims an aromatic diffuser apparatus with a replaceable cartridge for intermittently dispensing a fragrance or aroma into a surrounding area comprising an outer vessel having a base for positioning the diffuser on a horizontal surface and an open interior, the vessel being open at the top. A fan is located on the interior of the outer vessel with a heating element located within the outer vessel. There is a container located adjacent to the heating element, and the container is made of a heat conductive material which and comprises a means for holding a heated liquid which then releases the aroma from the heated liquid. There is also a wax member comprised of an aromatic or fragrant substance dispersed within a wax carrier, and the wax composition is located within the container The heating element partially melts the wax member so that an aroma or fragrance contained therein can be emitted and dispersed by the air flow generated by the fan. Therefore, the aromatic or fragrant substance is released upon heating to at least partially melt the wax carrier which can be solidified by cooling the device and upon the removal of heat trapping the aromatic or fragrant substance within the wax carrier to limit dispersion of the aromatic or fragrant substance;

U.S. Pat. Nos. 8,061,628; 8,210,448 and 7,722,807 to Kwvietok teach air fresheners for emitting two or more volatile fragrance compositions, wherein the device comprises a heater and wick which are configured to the first and second volatile compositions through said wick, and further, wherein said device comprises a timing mechanism configured to emit said first and second volatile compositions in a substantially continuous emission sequence in which the emission of the different volatile fragrance compositions automatically alternate between the different volatile compositions in the sequence, wherein in said alternating sequence: (a) said first volatile composition is emitted for a first time period, and said second volatile composition is emitted for a second time period, and the pattern repeats; again, and the pattern repeats; wherein said different volatile compositions are emitted during discrete emission periods, said discrete emission periods are each from 23⅓ minutes to less than 120 minutes in length.

U.S. Pat. No. 6,752,327 to Martens et al. discloses a plug-in air freshener having a piezoelectric actuator that vibrates a tilted orifice plate to dispense perfumes. The device includes a wick and a piezoelectric actuator which is physically coupled to an orifice plate. The coupled piezoelectric actuator may cause bimodal droplets of fragrance to deposit on the adjacent wall surface even with the titled orifice plate and the upwardly extending protrusion positioned between the orifice plate and the adjacent wall surface.

U.S. Pat. No. 6,960,625 to Christensen et. al. discloses and claims a transparent polyurethane—hydrogel composition formulated as an air-freshener application that comprises the reaction product of a pre-polymer and a water-soluble cross-linker in an aqueous solvent with little to no organic solvent. The pre-polymer is present in an amount of no greater than about 5.0 wt. % based on the total weight of all the hydrogel components. The pre-polymer is generally prepared from at least one water-soluble polyol and an isocyanate. The transparent polyurethane-hydrogel composition allegedly has desirable gel physical properties and is useful for an air-freshener application.

U.S. Pat. No. 6,294,162 to Semoff et al. teaches a transparent gel air freshener and method for its' preparation. The gel composition comprises an aqueous gel, a fragrance, a surfactant and a co-solvent. The gel is transparent and free from visible particles and is homogeneous throughout. It has a uniform texture, a continuous structure, and includes volatile scented components. The gel is also capable of suspending solids such as botanicals therein for a decorative effect. The method provides for preparation of a gel air freshener, including the suspension of botanicals therein, while maintaining the clarity, texture, and structure of the gel. The method includes the steps of preparing a gel composition and cooling the gel composition. Botanicals may be added to the gel composition when it has gelled enough to support the botanical on the gel surface

U.S. Pat. No. 7,166,259 to Beam et. al. and U.S. Pat. No. 5,695,692 to Kennedy are examples of commercially available plug-in or otherwise electric devices that automatically discharge a fragrance spray into a room for a given period of time. These often include a replaceable and re-fillable perfumed composition in a detachable bottle or container that when plugged in, emits a fragrance which is driven out into the surrounding atmosphere or immediate environs by an internally disposed electrical pump.

United States Published Patent Application No. 2011/0027124 to Albee et. al. discloses an electric fragrance diffuser including a housing having a plurality of outlet and inlet vents, a chamber for holding a fragrance medium, and a fan for drawing air from the inlet vents and creating an updraft airflow which facilitates the emission of the fragrance from the fragrance medium. The electric fragrance diffuser also includes a cowling structure for increasing and directing airflow in an upward direction. The cowling structure further interacts with inlet vents in the housing for accelerating the airflow in an upward direction and thereby accelerating the emission of the fragrance from the fragrance medium, wherein the fragrance is released from the apparatus via the outlet vents. The electric fragrance diffuser further comprises a storage compartment for storing at least one additional fragrance medium.

A known disadvantage of the fan-based systems is that they are incapable of dispersing high concentrations of the fragrance into the central area of a room. One effort to fix this problem was to include a louver structure, disposed upstream of the fan. The louver structure is generally angled upwardly and away from the fan for directing the airstream created by the fan upwardly and away from the upper portion of the fragrance container. Although this structure assists in directing the airflow of the fragrance, it does not affect the force of the airflow.

In addition, the aforementioned systems use containers that are prone to spillovers. Known air freshener systems generally contain a breakable fragrance bottle or cartridge that is exposed to the user at all times. Breakable fragrance bottles exposed to the user are prone to falling and subsequently shattering. Similarly, the cartridges may be disengaged, whereby the liquid may spill outside the air freshener. The liquid fragrance may drip on the floor or furniture and create a harmful, dangerous and/or unsanitary environment. In addition, these fragrance systems implement special containers that are made to fit the fragrance system and are only available through one supplier. As such, only a limited variety of fragrances is available, and if the supplier discontinues the item, the user is left with an unusable device.

In light of the shortcomings described above concerning fan-based fragrance diffusers, there is a clear need for a more efficient method of directing the airflow within the diffuser in order to maximize the efficiency of the diffuser as well as maximize fragrance dispersion. There is also a clear need for a fragrance diffuser that allows a user to utilize any essential oil via an absorption pad or fragrance medium system.

### II SUMAMRY OF THE INVEnTION

The present invention is an improved electronic fragrance diffuser with an improved air flow. The device utilizes a fragrance-containing gel, pad or polymer insert that contains a fragrance while the diffuser itself has an aesthetically pleasing design. A small electric fan is located within a centrally disposed chamber creates an airflow updraft suction effect, which directs and maximizes airflow through the fragrance medium in order to more efficiently release its' aroma. In this way, the fragrance diffuser disperses and continuously releases a high concentration of a selected air freshener or scent into the surrounding environs. The fragrance diffuser is not limited to certain fragrance containers or specific scents and also comprises an improved drawer or platform design which provides maximum air flow though the fragrance medium and prevents any liquid fragrance from escaping the housing. The unique housing design of the present invention enables the storage of additional unused fragrance mediums within the housing of the fragrance air freshener. These are then readily available to allow the user to easily change or replace a fragrance medium.

### III BRIEF DESCRIPTION OF THE DRAWINGS

FIG. 1a is an angled upper frontal view of an electric fragrance diffuser of the present invention.

FIG. 1b is an angled upper right side perspective view of the electric fragrance diffuser of the present invention.

FIG. 2a is an angled upper frontal view of an electric fragrance diffuser of the present invention designed as a cube.

FIG. 2b is an angled upper right side perspective view of the electric fragrance diffuser of the present invention designed as a cube.

FIG. 3 is a sectional side view of the inner components of the electric fragrance diffuser taken along lines 1-1 shown in FIG. 1a.

FIG. 4 is an opposite sectional side view of the inner components of the electric fragrance diffuser taken along lines 2-2 shown in FIG. 1b.

FIG. 5 is a top perspective view of the electric fragrance diffuser of the present invention designed as a cube

FIG. 6a is an angled top view of the top of the fragrance tray of the present invention.

FIG. 6b is a skewed lower view of the bottom of the fragrance tray of the present invention.

FIG. 7 is an exploded side view of the cylindrical embodiment of the present invention showing the internal components.

### IV. DETAILED DESCRIPTION OF THE INVENTION

The present invention comprises an improved electronic fragrance diffuser system with an enhanced flow. The air-freshening device may be designed in any one of a number of geometric three-dimensional shapes such as a cube, cone, cylinder, sphere or pyramid. Preferably, a cube design is utilized and the device will be hereinafter described from this perspective. Therefore, a detailed illustrative embodiment of the present invention is disclosed herein as a cube design. However, other structures and similar designs in accordance with the present invention may be embodied in a wide variety of forms and modes, some of which may be quite different from those in the disclosed embodiment.

The electric fragrance diffuser also includes a cowling structure for increasing and directing airflow in an upward direction. The cowling structure further interacts with inlet vents in the housing for accelerating the airflow in an upward direction through the fragrance pad and thereby accelerating the emission of the fragrance from the fragrance medium, wherein the fragrance is released from the apparatus via the outlet vents. The electric fragrance diffuser further comprises a storage compartment for storing at least one additional fragrance medium.

The device utilizes a fragrance-containing gel, pad or polymer insert that contains a fragrance while the diffuser itself has an aesthetically pleasing design that is compact and can be placed anywhere within room without being conspicuously evident. A small electric fan is located within a centrally disposed chamber and when it is turned on an airflow updraft suction effect is created that directs and maximizes airflow through a fragrance medium which then more efficiently releases its' aroma. The fragrance medium is placed on a uniquely designed drawer tray with multiple holes or pores which enable the fan-generated air flow to pass around and through the medium. The tray is slideably inserted into the air freshener device through a slot in a side wall comprising a part of the perimeter of the device, the preferred embodiment of which is a cube. The tray is planar in design and as will be seen is parallel to the top and base of the body of the cube. When pulled out and away from the main body of the diffuser, the air freshener composition, i.e., the pad, oil or polymer may be placed thereon. When the drawer tray is pushed back into the slot of cube, the fragrance source, i.e., the polymer or pad, is positioned over the multiple holes of the tray directly under the electric fan within the channel of air produced thereby. The fragrance is dispersed by the passing air draft up and out through holes in the top of the body of the cube and into the room wherever the air freshener is placed. A similar fan driven room air freshener is disclosed in U.S. Published Appln. No. 20110027124. also to Albee et. al. which is herein incorporated by reference.

The present invention then, is a fragrance diffusing apparatus comprising: a hollow housing consisting of a top dispersion cap with at least one outlet and a motorized fan disposed therein. Attached thereto is a central body portion having a plurality of first inlet vents and at least one outlet portal connected to said dispersion cap and a lower base portion connected to said central body portion containing an electric power source for running said motorized fan. All three sections are connected as an integrated unit so as to form a single, central cavity that is contiguous throughout. The central body portion further contains an open slot which is contiguous with and leads to said central cavity for the insertion of a fragrance tray.

The fragrance tray is substantially planar and is perforated to allow for the passage of airflow created by the by the fan above as it is drawn up from air inlet vents below. The peripheral boundary of said fragrance tray is elevated above the planar surface of said tray to create a trough for containment of the fragrance medium. The tray also has a locking tab disposed at its' distal end.

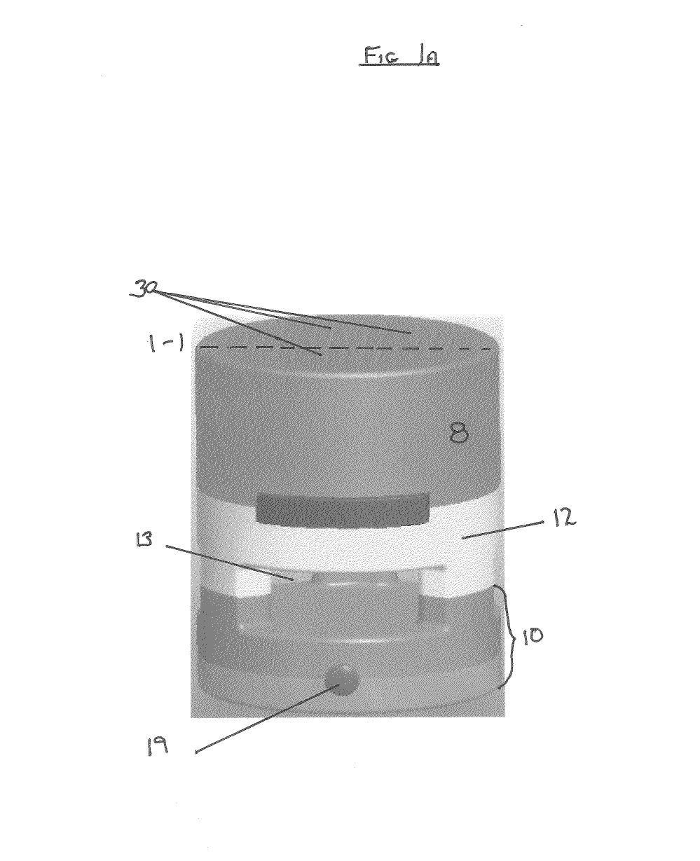

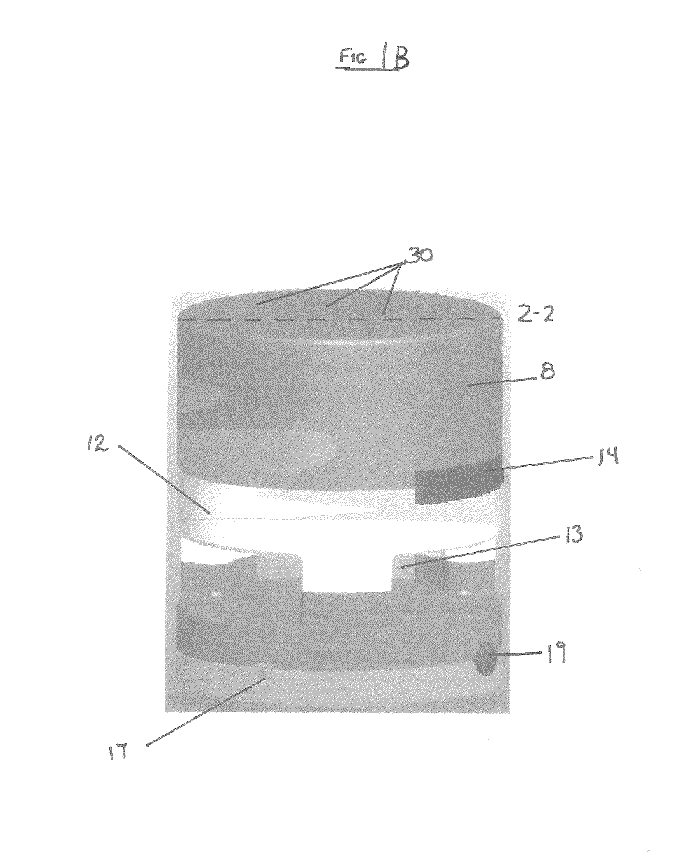

Referring now to FIGS. 1a and 1b, an entire cylindrical embodiment of an electric fragrance diffuser is shown from a frontal perspective view 2 of the present invention. The cube is comprised of three sections, the top 8, middle 12 and the square (or ellipsoidal) planar top 6 which is essentially perpendicular thereto as is a similar square base (not shown). The top half of the structure comprises a top housing 8 in which the electric fan is encased and below that is a base portion 10 with a substantially open middle section comprising the inlet vents 12 disposed there between. The base 10 generally will encase the power source for the motor (not shown) that drives the fan encased in the top housing portion of the fragrance dispenser 2.

Referring now to FIG. 1b the fragrance dispenser of the present invention then, consists of a main body 2 comprising an upper housing fan unit 8 and a base power unit 10 with air inlet apertures or portals 12 disposed there between. The upper housing unit 8 encases not only the fan and electric motor but a drawer slot 14 below the fan for holding a tray-containing fragrance medium. The drawer 14 consists of a slot portion 16 (not shown) within the body of the air freshener with a sideably—removable drawer 22 that can be pulled out from the housing to allow for the replacement of the fragrance medium. Directly above this chamber, an electric fan (not shown) is located that, when turned on, creates an upstream airflow (arrows) drawing the outside air and facilitating the emission of the fragrance from the fragrance medium, the airflow entering the inlet vents and exiting the outlet vents. The housing further contains a cowling structure that increases airflow and directs airflow in an upward direction. In this way the cowling structure directs and accelerates the emission of a fragrance from the fragrance medium to the surrounding outside environment.

The air freshening device of the present invention also comprises an on/off switch 15 in the base 10 of the cube which, when pushed in or depressed into the base completes the electrical circuit and turns on the electric motor 21 of the fan 25. On an adjacent side of the base, an AC adapter outlet 17 allows for an alternate power source for the electric motor should batteries be unavailable. The unique housing design of the present invention enables the storage of additional unused fragrance mediums within the housing of the fragrance air freshener. These are then readily available to allow the user to easily change or replace a fragrance medium, by removing the used “spent” medium from the tray and replacing it with a fresh one.

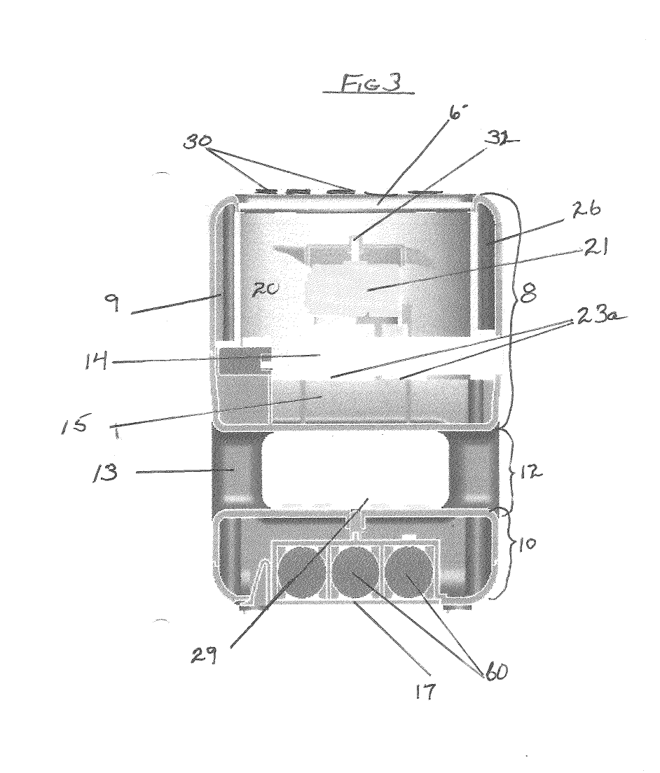

Referring now to the cut-away view of FIG. 3, the cube embodiment of the present invention 2 comprises the top portion 8 consisting of a housing 9 that contains an electric fan 21 for producing the ventilated “draw” of air for dispersing a desired fragrance through holes or apertures in the top surface 6, a central or middle portion 12 consisting of the air inlet ducts 13 and a substantially planar slot 16 for inserting the fragrance medium tray and a lower or bottom portion 10. The top portion housing further contains a cowling structure 26 within that increases airflow and directs it in an upward direction. In this way, the cowling structure 26 directs and accelerates the emission of a fragrance from the fragrance medium.

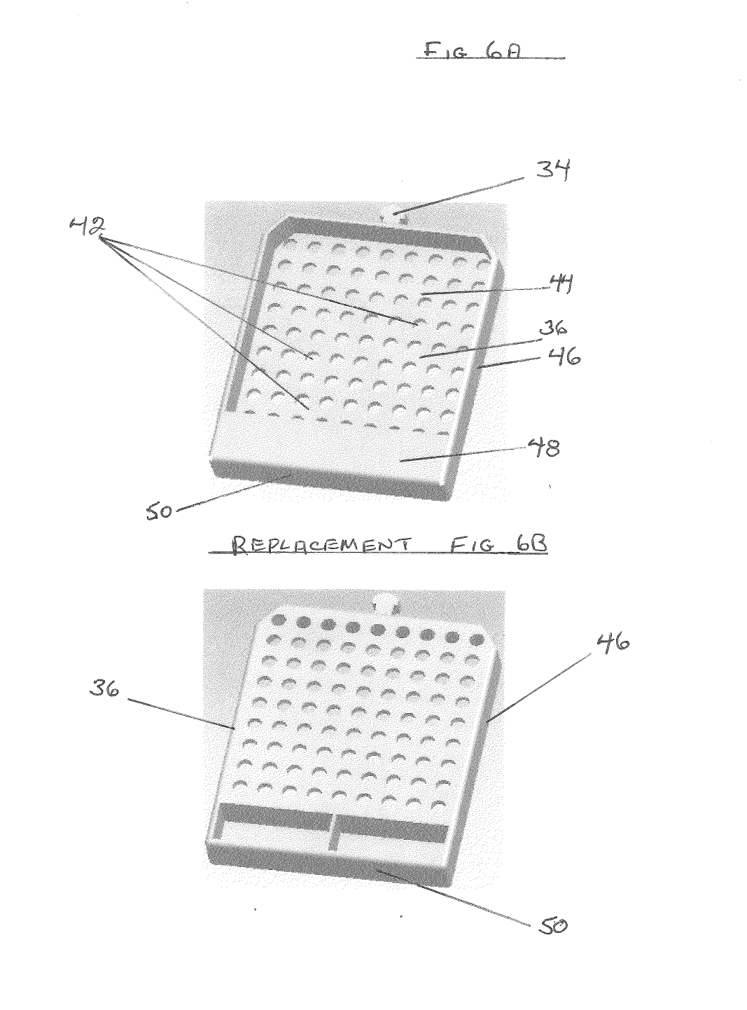

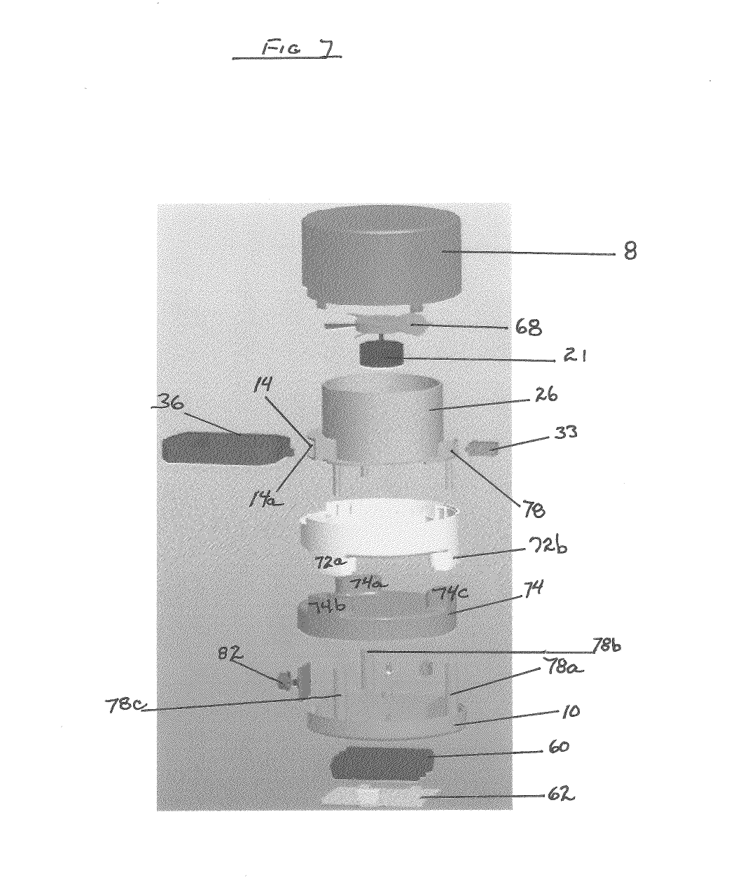

The middle section 15 contains the slot 16 which holds a slideable tray (not shown), that is preferably removable from the housing to allow for ease of replacing the fragrance medium. Once a new fragrance medium is placed into the tray, the tray may then be pushed back into the slot 16 of the air freshener body 2. The slot is also designed with a series of vents 23a, 23b which, when the fan is turned on, provides multiple passage ways to create a flow of air (arrows) from the outside through the air inlet apertures or portals 12 and up through the cowling 26 and the fragrance-containing tray (FIG. 6). The tray is perforated substantially throughout, thereby containing many small holes that allow the air-flow generated by the fan 21 to pass through. The fragrance medium is preferably made from a permeable material capable of absorbing a scented material fragrance such as a fragrance oil and more specifically may consist of a fragrance absorption pad, a fragrance—saturated felt pad, a scented solid, a membrane gel, potpourri, or ceramic scented beads. The tray then contains multiple perforations or holes, which with the fan, enables for the creation of an upstream airflow and facilitates the emission of the fragrance from the fragrance medium, the airflow entering inlet vents and exiting the outlet vents.

The cowling chamber (26) for holding the fragrance medium optimally contains at least one aperture (28) for facilitating the release of fragrance from the fragrance medium and a tab for coupling the chamber to the apparatus. The apparatus may also include a storage compartment removably attached to the housing. The storage compartment of the present invention selectively couples to the housing and preferably contains at least one unused fragrance medium.

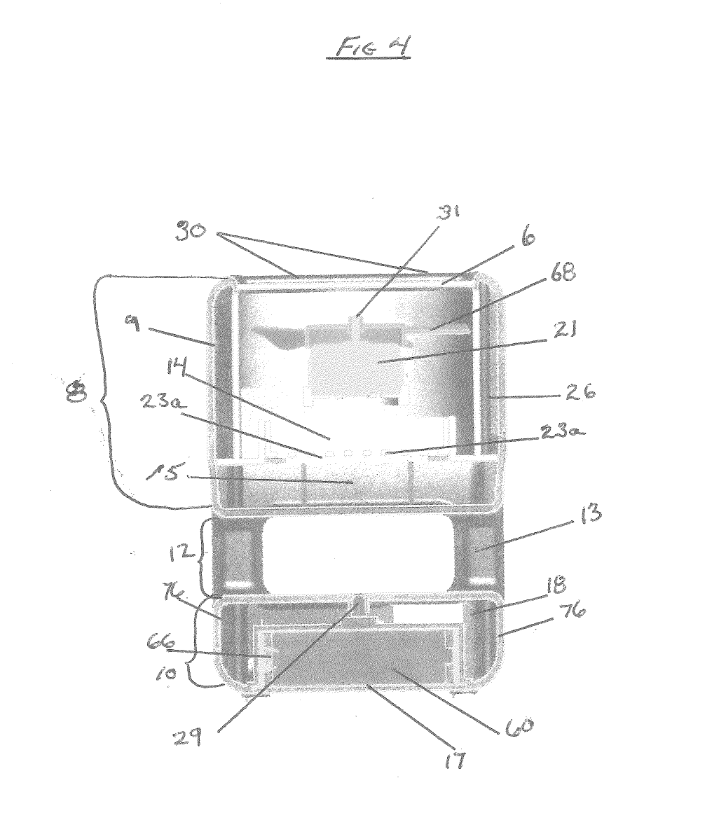

Referring now to FIG. 4, the electric air-freshening device of the present invention comprises a multi-part housing comprising a top section 8, a middle portion section 12, and a base or bottom section 10 with a power source 17 within a base compartment 18. The three sections are preferably constructed from any readily moldable sturdy plastic known in the art. Preferably, the top section 8, the middle section 12 and the base section 10 with the power source 17 are molded as separate units and glued or fused together as is known in the art. The base section may further comprise a storage compartment (not shown) and each are formed from injection molded plastic such that they comprise a single molded piece. Each section may also be molded together as one to form a continuous, single cube or cylinder housing design as is known in the art. It is to be recognized that although the electric fragrance air-freshening embodiments shown are as a substantially cubic form, any geometrical shape or design such as a cylinder, sphere or pyramid may be designed providing it is practical from a production and economical standpoint.

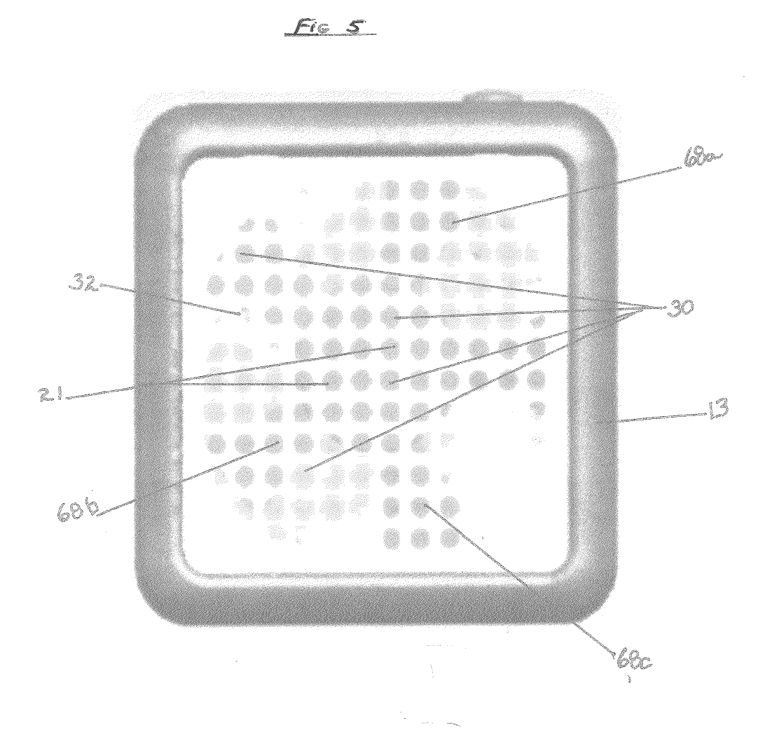

As shown in FIG. 5, the top section 13 is essentially a planar square by design with a spherically arranged plurality of holes or apertures 30 which serve as outlet vents for the air flow generated from below within the cube by the fan. These multiple outlet vents 30 primarily allow for the release of the aroma or fragrance from the air-freshening device 2 when in operation. The outlet holes 30 release the fragrance upward into essentially a 360° circular dispersion into the surrounding atmosphere. Whereas, taken as a whole, the multiple outlet apertures are preferably arranged to form a pattern on the top section 13 that is substantially circular in shape, the outlet apertures or holes 30 may be any appropriate shape or size and may be in any appropriate and/or visually aesthetic design on the top piece 13 of the upper section 8 of the device. Furthermore, any appropriate number of outlet vents or holes 30 may be implemented in the design of the top piece.

Looking down through the outlet apertures one can see and get a visual perspective on the location of the underlying fan blades 68 and the centrally positioned motor 21. The top section 8 also comprises the main slot or insert groove 20 for the insertion of the fragrance tray(not shown) 6 that is removeably inserted into the body of the air freshening device so that the fragrance composition contacts the air flow provided by the fan 21, 25 when it is turned on. The tray perimeter is flush with the four (4) walls of the insides of the air freshening cube or cylinder when inserted.

Referring now to FIG. 5, the fragrance tray is shown which is also comprised of multiple pores or holes 42 that extend throughout the trays' thickness, i.e., the substantially flat and planar body of the tray 36. Whereas FIG. 5a shows the top of the tray looking down, FIG. 5b shows the tray from a perspective underneath The multiple number of pores 42 insure the maximum amount of contact between the air current generated by the fan and the fragrance material (pad, polymer, wax or gel) when placed thereon. The holes are preferably arranged in parallel, vertically and horizontally throughout the planar face of the tray although any arrangement is possible and one of no logical pattern is certainly within the scope of the present invention. The greater the number of holes 42 in the tray, the greater the rate of diffusion of the fragrance into the air flow that is generated by the fan and emitted into the surrounding room or space wherein the air freshening device is placed. The fragrance material is situated directly under the fan in the air flow that is drawn inward through the inlet portals in the base when the tray is fully inserted in the body of the device 2.

Referring now to FIG. 3, the interior of the air freshening device of the present invention is seen as a transverse sectional view, cut along the lines 1-1 as shown in FIG. 1. The top 8, middle 12 and bottom 10 sections can be seen in their positioned physical perspectives. Section 8 might be generically regarded as the motor/fan section, 12 as the intake section and 10 as the power source base. Here the power source is shown as side view of the section 10 with one or more batteries 60 with an anode (+) and a cathode (−) end. When inserted into the air freshening unit, the batteries fit into a cartridge containment portion 62 in the base wherein positive and negative contacts are made to complete an electrical circuit 64a, 64b, that runs throughout the body 2 of the device and drives the fan motor 21. A spring contact 66 at one end of the compartment conducts the electrical charge and forcibly presses against the positive (+) end of the battery and holds it in place. Again, in another embodiment of the present invention, the batteries may be replaced by a coil from a DC outlet and plugged into a wall outlet accordingly.

Once the air freshener unit 2 has been properly prepared for activation through the insertion of the necessary batteries and air freshener compound as described above, the user may then place the unit 2 on a stable surface. To activate the freshener, the user may preferably press switch 19 (see FIG. 1) whereby light source 70 becomes activated and lights up. The motor 21 revolves the fan blades 68 creating an upward air flow from the inlet vents 13 and the unit releases a selected preferred fragrance from the pad or gel that has been placed into the tray 36 into the air. In other words, during operation, the air fragrance diffuser 2 is energized by receiving electricity from the batteries when the electrical circuit is completed by pressing the on/off switch 19. Electrical power is drawn from the batteries 60 to fan motor 21 and light source 70 by means of the electrical conducting wire 76 and basic electrical components (not shown) commonly used in the art. When turned on, the fan creates an air flow which passes though the slots 23 or apertures or air inlet vents and then up through the cowling 26 of the housing and then up through the air outlet vents which are substantially circular in shape and are disposed right above the fan in the top section 8.

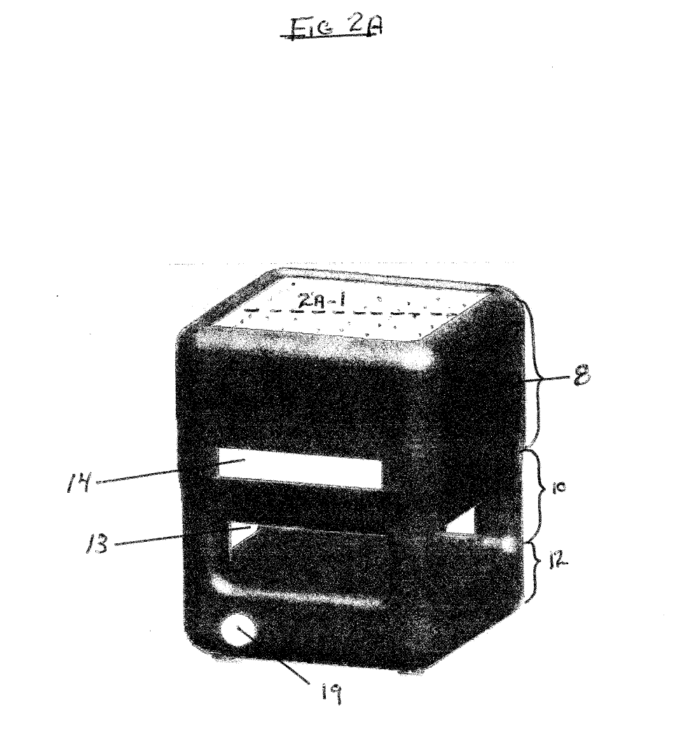

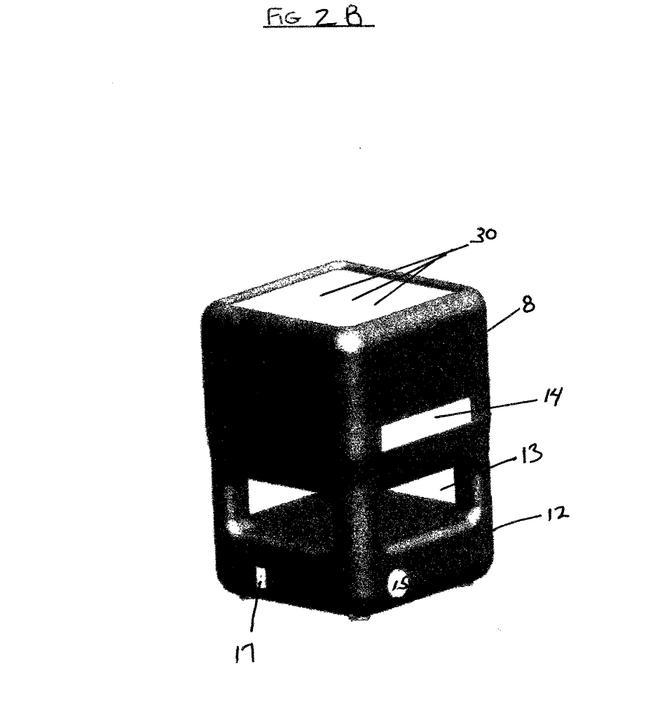

Specifically, air inlet vents 13 encircle the perimeter of the middle section 12. The air inlet vents allow air to be pulled into diffuser 2, which assists in increasing the airflow velocity and directing the airflow into and through the fragrance diffuser. Additionally, any appropriate number of air inlet vents 15 may be implemented in the design without limiting the scope of the present invention. As shown in FIGS. 1 and 2, middle section 12 is rectangular in shape and preferably can receive a fragrance tray, a drawer 16, and fan 21, 22. The fan 21, 25 is preferably a standard fan commonly used in the art, whereby the fan comprises a hub or drive shaft 32, a plurality of fan blades 25, and a motor 21 disposed thereunder. The motor 21 is circular in shape and the fan blades 25 are circumferentially disposed around the top circumference thereof. The proximate end of each fan blade 27 is preferably molded to the driveshaft hub 32, whereby the distal end of each fan blade 25 extends outwardly from the hub 32. In order to facilitate the rapid rotation of fan blades, the driveshaft 32 is preferably located on top of the motor 21 and the driveshaft 32b and is rotatably—coupled to a center axel (not shown) of the motor as commonly known in the art. Fan 21 and fan blades 25 are preferably constructed from a single piece molded plastic. However, either component may be constructed from any appropriate material without limiting the scope of the present invention.

The middle section 8 of the unit comprises the cowling 26 which is preferably constructed from plastic and is coupled on its lower edge 29 to the outer wall of the middle section 14. Moreover, the cowling 26 is preferably coupled to the outer wall through a method of molding plastic as commonly known in the art. As shown in FIGS. 3 and 4, the cowling completely encloses the fan 21 , and the sides of the cowling 26 is located in close proximity to the tips of fan blades 25. The fan blades are substantially enclosed by the cowling and this maximizes the fan's 21 efficiency as the it increases the velocity of the airflow from the vents below.

Referring now to FIG. 5, the fragrance tray 36 is comprised of an upper side 44 (as depicted in FIG. 5a) and a lower underside (as shown in FIG. 5b). Referring first to FIG. 5a, the upper side of the tray 44 is where the fragrance material (pad, polymer, wax or gel, not shown) is actually placed. The tray is comprised of the planar retention portion 44a which contains the multiple pores or holes 42 through which the air flow generated by the fan passes. The tray 44 is generally square or rectangular in shape (although it is not required or limited thereto) and the four (4) sides thereof have walls 46 extending several millimeters above and entirely around the periphery of the tray. The distal end of the tray 48, that is to say, the end that is last pushed into the slot 20 of the body 2 of the air freshening device, is comprised of a wall 50 that is substantially thicker than the other three walls of the tray. This is formed as a trough-like structure and enables the user or consumer to grasp the tray when pulling it out and/or pushing it in. The exterior side wall of the tray 50 itself becomes flat against, and flush with the external wall of the top portion 8 when closed so as to essentially appear to be a part thereof.

Again referring to FIGS. 3 and 4, top section 8 further comprises an opening 16 and a cavity 20. The opening 16 is large enough to slide-ably receive the fragrance tray 36 which when placed therein, enters the opening and the cavity 26 which receives and retains the tray 36. Specifically, the top section 8 preferably comprises a cavity 20 disposed there within. The cavity 20 houses a coupling member 33 for engaging a tab 34 in the fragrance tray 36. Coupling member 33 engages and secures the arrow-head shaped prong of the tab 34 in the proximal end of the tray. Preferably, the coupling member 33 receives the tab 34 attached to the tray 36 in order to couple the tray and secure it to the cavity side walls 26 of the air freshener body 2. Once a user slides the tray 36 into the cavity of the unit, the user may then push front surface 48 of the tray 36 so that the tab 34 fits within prongs (not shown) of the coupling member 33. The user can continue to push the front surface 48 so that the tab 34 activates the spring-loaded mechanism of coupling member 33 whereby tab 34 becomes locked into place and the tray 36 subsequently remains disposed within the cavity 26 of top section 8. In order to release tab 34 from the coupling member 33, the user can push front surface 48 thereby releasing the spring-loaded mechanism within coupling member 33. Once tab 34 is released from coupling member 33, the drawer is pushed outward by the spring-loaded mechanism and the user may remove the tray 46 from the cavity 20. While a specific method and mechanism is described for selectively inserting and coupling the tray 36 within air freshener device 2, it should be appreciated that any method and mechanism commonly known in the art may be used without limiting the scope of the present invention.

As further shown in FIG. 5, fragrance—retention tray 36 comprises a plurality of apertures or holes 42 formed in the seat 44 of the tray. When using the present invention, a user places a fragrance medium (pad, polymer, wax or gel, not shown) in the tray. The fragrance medium is preferably made from a permeable material capable of absorbing fragrance, such as fragrance oil. Once the fragrance—retention tray 36 is disposed within middle section 10, air-flow portals 23 allow air to enter and pass through the bottom of the tray to facilitate the release of a fragrance into the air. The air-flow passes over and about the volatile scent components of the fragrance which evaporate therein. The fragrance medium may also consist of a fragrance absorption pad, a saturated felt pad, a solid scented piece, a membrane gel tray, potpourri, ceramic scented beads, or any appropriate medium commonly known in the art. Fragrance medium may be made of cotton, polyester, a polymer or plastic or any appropriate material without departing from the spirit of the present invention. The fragrance medium may be impregnated with any fragrance oil commonly available on the market for use with fragrance oil burners or diffusers. The fragrance, oils may be purchased from any supplier and are available in a wide variety of fragrances. Moreover, since this configuration allows the fragrance oil to be stored hidden within the diffuser, the electric fragrance diffuser of the present invention eliminates spills and potentially hazardous conditions created by other fragrance diffusers.

Bottom section 12 also preferably contains an on-off switch . Preferably, as shown in FIG. 1a activation switch 19 is a push button design, whereby the electric air diffuser 2 is activated when a user pushes in or depresses switch 19. However, it is to be understood that switch 19 may be any type of switch commonly known in the art (i.e., an on/off push button switch, an on/off sliding switch, a light sensing, optic activation switch, etc.) without limiting the scope of the present invention. Additionally, activation switch 19 may also activate a light source 29 whereby light source 29 may be any type of light source commonly known in the art, such as a light emitting diode. Accordingly, once a user depresses switch 19, light source 29 becomes active and remains active for as long as switch 19 is on. Thus, light source 29 serves to notify the user that air diffusion device 2 is currently active. It should be appreciated that light source 29 may be located anywhere within the surface of the diffuser body 2 without departing from the spirit of the present invention. It should also be appreciated that light source 29 may provide additional functions such as a battery life indicator.

Referring now to FIG. 7, the air freshener device of the present invention is shown as an exploded side view prospective. The top cap portion 8a which comprises the outer housing of the unit fits down and over the fan blades 68 and the motor 21 which then fit into the housing cowling 26 which fits into and is secured to the cap 8a . The fragrance—retention tray 36 is shown in juxtaposition the left side of the housing cowling 26 in a position the lies removed from and just outside the entry slot 14 into which, in practice, the tray is pressed when loaded with and contains the fragrance-containing medium. Circumferential flanges 14a surround the slot 14 and these enable the user to grasp the sides of the tray 36 for removal thereof from the housing unit so the air freshener medium (gel, liquid, etc.) can be replenished as necessary.

Also disclosed in FIG. 7 is the coupling member 33 shown separate and apart from the housing cowling 26 into which it is inserted when the air freshener device 2 is first assembled. Like the tray 36 to which it is clamped when the tray is operationally inserted, the coupling member 33 fits into a smaller slot 70 that is positioned directly opposite to and in line with the entry slot 14 which holds the fragrance—retention tray 36. The “clamp—slot” so to speak 70 is smaller in size than the tray—holding slot although similarly rectangular in shape to match that of the four sides of the coupling member 33 so as to be flush thereto when inserted. The clamp—slot is also provided with circumferential flanges 72 which surround the slot to allow ease of insertion and removal of the coupling member 33. Again the shape of these elements is not limited to rectangular or square and any shape is contemplated within the spirit and scope of the present invention so long as the pieces fit hand—in—glove.

Two housing wall units 72 and 74 comprise the middle section thereof when fully assembled. These then form the air intake ducts 76 described in detail earlier. Outside air is drawn in by the operation of the fan 21 in the top housing 8a . Air entering the cowling is compacted as it moves up and though the cowling 20 of the air freshener housing 2. The power base section 10 is comprised of a housing ring 78 with three (3) equidistantly disposed posts 78a , 78b and 78c arising therefrom. The ring section 10 forms a base while the posts 78a , 78b and 78c are a part thereof and formed therefrom. The posts 78a , 78b and 78c are inserted up and into protrusions 72a , 72b , and 72c which form the walls of the middle housing wall units 72 and 74 when fully assembled and anchor the entire air freshening device together. These posts also serve as conduits for the electrical wiring system (not shown) that connects the power source such as the batteries 60 or external AC source to the on/off switch and the electric motor driven fan located in the top housing section of the unit. The center of the base 80 is configured as a trough or well that is preferably rectangular in design in order to accommodate two or more AA or AAA batteries which serve as the power source for the motorized fan located in the top housing section 8 above the cowling 26. If batteries are employed as the power source, the on/off button or switch 19 when pressed inwardly engages the anode/diode contacts of said batteries through the conducting element 64 discussed previously which completes the circuit as is known in the art.

When the unit disclosed in FIG. 7 is fully assembled and operational, the batteries 60 are inserted into the base 10 through an open portal therein (not shown) and maintained in position by a removable door 62 which snaps into place as is known in the art. Once engaged, the electric motor and fan 21, 68 can be turned on thereby creating a powerful updraft which disperses the fragrance contained above throughout the room.

The present invention is best understood by referring to the alternative embodiments set forth and referred to above in the accompanying figures. It is to be understood however, that the illustrated embodiments set forth in said figures, (as well as their operation) are merely examples of ways and means for carrying out the present invention and none of these are intended to limit the spirit and scope of the present invention as defined and set forth in the claims that follow.

What we claim is:

1. A fragrance diffusion device comprising a hollow housing consists of:
i. a top dispersion cap with at least one outlet and a motorized fan disposed therein;
ii. a central body portion having a plurality of first inlet vents and at least one outlet portal connected to said dispersion cap;
iii. a lower base portion connected to said central body portion containing an electric power source for running said motorized fan, and
iv. where all three sections are connected as an integrated unit so as to form a single, central cavity that is contiguous throughout.

2. The fragrance diffusing apparatus of claim 1 wherein said central body portion contains an open slot for the insertion of a fragrance tray.

3. The fragrance diffusing apparatus of claim 2 wherein said open slot is contiguous with and leads to said central cavity.

4. The fragrance diffusing apparatus of claim 3 wherein said fragrance tray is substantially planar and perforated for the passage of airflow with a locking tab disposed at its' distal end.

5. The fragrance diffusing apparatus of claim 4 wherein said fragrance tray is substantially planar with walls about its' outer periphery designed to hold a fragrance medium for the passage of airflow there-through

6. The fragrance diffusing apparatus of claim 4 wherein said open slot is positioned to receive the insertion of said fragrance tray so that the tray is centrally disposed within said central cavity.

7. The fragrance diffusing apparatus of claim 6 wherein the peripheral boundary of said fragrance tray is elevated above the planar surface of said tray to create a trough for containment of said fragrance medium.

8. The fragrance diffusing apparatus of claim 7 wherein the proximal end of said fragrance tray is substantially the same size as the outer perimeter of the slot leading to the central cavity.

9. The fragrance diffusing apparatus of claim 8 wherein the locking tab disposed at the distal end of said fragrance tray engages with a locking clip in the wall of said central cavity.

10. The fragrance diffusing apparatus of claim 9 wherein the proximal end of said fragrance tray is substantially flush with the outer perimeter of the slot leading to the central cavity and the outer wall of the central body portion when the fragrance tray is inserted therein and the distal end of said fragrance tray is engaged with the locking clip in the wall of said central cavity.

11. The fragrance diffusing apparatus of claim 10 wherein the electric power source for running said motorized fan comprises batteries or a DC jack that can be wired for direct wall plug in.

12. The fragrance diffusing apparatus of claim 11 wherein the fragrance medium is selected from the group consisting of a fragranced pad, fragranced gel, a liquid gel pack or fragranced polymer insert.

13. The fragrance diffusing apparatus of claim 12 wherein outside air external to the walls of the apparatus is drawn in through the first inlet vents and driven into the central cavity creating maximum air flow thru and around the fragrance medium.

14. The fragrance diffusing apparatus of claim 13 wherein said central cavity forms a substantially conical cowling within the central body portion of the unit.

15. The apparatus of claim 14 wherein said fan draws air into said cowling within the housing via said plurality of first inlet vents and thereby increases the airflow velocity and facilitates the emission of said fragrance from said housing through the top dispersion cap.

16. The apparatus of claim 15 wherein said plurality of first inlet vents are disposed above said plurality of second inlet vents forming a space there between which contains a spring-loaded, fully removable fragrance tray disposed in said space adapted for retaining a fragrance medium with a generally vertical cowling structure disposed above said chamber comprising a substantially cylindrical vertical wall disposed in close proximity to said plurality of first inlet vents; a fan disposed within said cowling structure comprising a plurality of fan blades substantially encased in said cylindrical vertical wall wherein said fan creates an airflow for dispersing said fragrance; and, a storage compartment remove-ably attached below said housing for storing at least one additional fragrance medium.

17. The apparatus according to claim 16, wherein said fan and said cowling structure draw air into said housing via said plurality of first inlet vents to facilitate the emission of a fragrance from said fragrance medium.

18. The apparatus according to claim 17, wherein said fan and said cowling structure draw air into said housing via said plurality of first inlet vents for increasing airflow velocity and facilitating the emission of said fragrance from said housing.

19. The apparatus according to claim 18, wherein said fragrance medium is a permeable material capable of absorbing a liquid, volatile fragrance.

20. The apparatus according to claim 19 wherein said fragrance medium is a fragrance absorption pad, a saturated felt pad, a scented solid, a membrane gel tray, potpourri, or a plurality of ceramic scented beads.

21. A fragrance diffusion apparatus comprising a housing that consists of a first section that has at least one primary aperture and at least one secondary aperture; said at least one primary aperture allows air to enter said housing and said at least one secondary aperture that allows air to exit said housing; a second section coupled to said first section, said second section including a fan and an airflow directing and accelerating cowling that has a slot to receive a spring-loaded, fully removable tray for holding a fragrance medium; a third section coupled to said second section, said third section including at least one aperture which allows air to enter said housing; and, a fourth section coupled to said third section, said fourth section including a storage compartment remove-ably attached to said fourth section for storing at least one additional fragrance medium.

22. An air fragrance diffusion apparatus, comprising: a hollow housing including at least one outlet vent, at least one first inlet vent, and at least one second inlet vent; a spring-loaded, fully removable chamber disposed in said housing for retaining a fragrance medium; a fan disposed in said housing for creating an airflow and facilitating the emission of a fragrance from said fragrance medium, said airflow entering said at least one first inlet vent and exiting said at least one outlet vent; an airflow directing means disposed above said chamber for increasing and directing said airflow in an upward direction; an air accelerating means for further increasing the velocity of said airflow via air entering through said at least one second inlet vent; and a storage compartment remove-ably attached to said housing for storing at least one additional fragrance medium.
